(19) United States
(12) Patent Application Publication
(10) Pub. No.: US 2007/0190173 A1
(43) Pub. Date: Aug. 16, 2007

(54) Antioxidant skin compositions and methods of production of the same

(76) Inventors: Roger D. Blotsky, Goodyear, AZ (US); Leslie A. Blodgett, San Francisco, CA (US); Ramon Figueroa, Hollywood, FL (US)

Correspondence Address: TOD R NISSLE, PO BOX 55630, PHOENIX, AZ, 85078, US

(21) Appl. No.: 11/638,311

(22) Filed: Dec. 12, 2006

Related U.S. Application Data
(63) Continuation-in-part of application No. 11/472,536, filed on Jun. 21, 2006.
Application No. 11/472,536 is a continuation-in-part of application No. 10/725,729, filed on Dec. 2, 2003.

(51) Int. Cl. A61K 33/24

(52) U.S. Cl. 424/617

(57) ABSTRACT

Antioxidant dry powder compositions are produced and topically administered externally to skin to enhance the ability of the skin to combat free radicals.

This is a continuation-in-part of U.S. patent application Ser. No. 11/472,536, filed Jun. 21, 2006, which is a continuation-in-part of U.S. patent application Ser. No. 10/725,729, filed Dec. 2, 2003.

This invention pertains to exfoliating cosmetic compositions and methods for producing the same.

More particularly, this invention pertains to a method for producing powdered exfoliating compositions that can be applied during day or night and include an unusually large number of naturally occurring minerals.

In a further respect, the invention pertains to a powdered exfoliating cosmetic mineral composition that is a leave-on daytime or nighttime product.

In another respect, the invention pertains to powdered exfoliating cosmetic mineral composition that yields an acidic pH when partially or fully in solution, and does not irritate dermal tissues when applied thereto.

In yet another respect, the invention pertains to powdered exfoliating cosmetic mineral compositions that include a significant number of mineral elements for application to skin.

The invention also pertains to skin compositions containing an unusually large number of naturally occurring minerals which have antioxidant properties.

Most particularly, the invention pertains to a method of producing antioxidant skin compositions that can be applied topically or on the skin.

In another respect, the invention pertains to antioxidant skin cleansing compositions that create cleansing, exfoliation, and skin conditioning benefits.

In yet another respect, the invention pertains to antioxidant skin cleansing compositions that can be produced in anhydrous powdered form that are substantially free of water to preserve the antioxidant potential of the composition.

The following definitions are utilized herein.

Chemical element. Any of more than 100 fundamental metallic and nonmetallic substances that consist of atoms of only one kind and that either singly or in combination constitute all matter, most of these substances lighter in weight than and including uranium being found in nature and the rest being produced artificially by causing changes in the atom nucleus.

Clay. A natural or synthetic colloidal lusterless earthy composition that includes tiny sheet-like layered particles of alumina and/or silica that are less than about 0.002 millimeters in size, that is generally plastic when moist, and that, when naturally occurring, includes decomposed igneous and/or metamorphic rocks. Most clays have a pH in the range of about 4.5 to 8.5. Natural and synthetic clays include mineral elements. Clays can, in additional to having particles less than five microns in size, include particles having a size greater than five microns.

Leonardite. A soft, loose-textured coal that has low BTU value. Leonardite is a humate; can include up to 70% by weight minerals; can be formed from lignite; can occur naturally as the result of not being heated and pressurized over time to the extent necessary to produce anthracite, lignite, or bituminous coal; and, can include compost as a component.

Mineral. Any naturally occurring chemical element or compound. A mineral has a characteristic crystal structure and chemical composition or range of compositions.

Mineral element. A chemical element that occurs naturally as or in a mineral. A mineral element may be produced using synthetic or manufacturing processes; however, each mineral element does occur naturally as or in a mineral.

Rare earth or rare earth element. Any one of a group of metallic elements with atomic numbers 58 through 71, including cerium, praseodymium, neodymium, promethium, samarium, euro0pium, gadolinium, terbium, dysprosium, holmium, erbium, thulium, ytterbium, and lutetium. In nature, rare earth elements are bound in combination with nonmetallic elements in the form of phosphates, carbonates, fluorides, silicates, and tantalates.

Sand. A loose material consisting of small but easily distinguishable grains usually less than two millimeters in diameter and more than about 0.02 millimeters in diameter, most commonly of quartz, resulting from the disintegration of rocks.

Silt. Unconsolidated or loose sedimentary material whose constituent rock particles are finer than grains of sand and larger than clay particles, specifically, material consisting of mineral soil particles ranging in diameter from about 0.02 to 0.002 millimeters.

Mineral elements are essential to life. The body however does not manufacture a single mineral element although all tissue and internal fluids contain them from bones, teeth, soft tissue, muscle, blood and nerve cells. The usefulness of mineral elements and of trace mineral elements in biological systems has been scientifically and medically established. Their complimentary function for enhancing nutrient exchange, improved conductivity of cellular transport, support essential osmotic balance of every tissue, fluid, cell and organ, and play a role on everything from muscle response, to transmission of messages through the nervous system, the production of hormones, digestion, and utilization of nutrients. They play a significant role in disease prevention not only in the functions described above, but on a genetic fundamental level, as biological systems require mineral elements to effectively and accurately program DNA synthesis required for cell replication. Any defective programming in DNA synthesis by deficient mineral element function could lead to abnormal replication and alternatively promote disease state or death.

The presence in the body of many mineral elements is the result of supplementation through diet. Macro mineral elements are those that the body requires in greater quantities than 100 mg daily, while Micro mineral elements are those that the body requires less than 100 mg daily. Food consumption, particularly of fruits and vegetables, is the only means to supplement vital mineral elements to the body. The introduction of processed food and the insurgence of soil mineral depletion have created a food market less apt to derive and deliver the mineral element requirements that were once delivered only by consumption. Today's synthetic vitamin and mineral element supplement market (which is valued in the billions of dollars) has been established on the basis that the human body is not getting all the necessary mineral elements through normal food consumption.

Soil depletion phenomena are real and measurable. Restoration of soil involves methods of crop re-cycling and use or organic fertilizers to help reconstitute the mineral content of soil. The use of organic fertilizers has been increasing in usage over the last three decades. Their increased usage is the result of environmental and agricultural concerns for moving towards a chemical-free and pesticide-free method of crop production coupled with a means for replenishment that can alleviate the soil depletion of minerals on farms overburdened by decades of use.

Soil taxonomy and the many sub-classifications yield earth matter that collectively includes all known natural minerals. Soils vary in their mineral content with some having predominant concentration a certain minerals and trace minerals. The minerals can be concentrated from the soil using extraction techniques known in the art and are usually identified and quantified by analytical equipment.

In all cases, soil classification and the extraction techniques applied to capture or recover minerals are the limiting factors in maximizing the total number and amounts of minerals identified and quantified. Most extraction techniques fail to capture a wide spectrum of inherent minerals found in soil.

One facet of the invention pertains to extraction techniques used to gather, isolate, and concentrate specific mineral elements. For example, U.S. Pat. No. 4,150,093 Kaminsky and U.S. Pat. No. 3,990,885 Baillie describe hot water extraction of tar sands yielding heavy minerals at specific high concentrations of titanium and zirconium.

Clay soil is one of the three principal types of general soil classifications, the other two being sandy soil and loamy soil. Most soils include silt.

The extraction techniques described herein relates in part to specific soils and soil combination compositions having taxonomic classifications including clay soil, sandy soil, and/or clay-sand soil comprising a combination of clay soil and sandy soil. Sandy soil typically is described as silicates. Soils classified as clay soils contain a significant percentage of clay in their composition, typically at least twenty percent by weight.

Soil includes very coarse, coarse, fine, very fine, and medium size particle sizes. The coarse particles range in size from 0.5-1.0 mm. The fine particles are from about 0.10 mm to 0.25 mm in size. The medium particles are from 0.25-0.50 mm in size. Very coarse particles are greater than about 1.0 mm in size. The very fine particles are less than about 0.10 mm in size.

The percent sand in clay-sand soil typically by definition equals or is greater than 20% by weight. The percent of silt in clay-sand soil typically by definition equals or is greater than 20% by weight.

Two samples of selected soil were analyzed by A&L laboratories in Memphis, Tenn. with the following results:

| Soil Sample | Classification | % Clay | % Sand | % Silt |
|---|---|---|---|---|
| Site # 4 | Clay | 22.5 | 36.5 | 40.9 |
| Site # 5 | Clay | 23.1 | 24.4 | 52.5 |

The soils from Sites 4 and/or 5 or other sites were collected and subjected to the aqueous extraction process described below to produce both a liquid mineral element composition containing mineral elements and to produce a dry powder mineral element composition. The dry powder mineral element composition is produced by drying the liquid mineral element composition.

Both the liquid mineral element composition and the dry powder mineral element composition capture and recover similar mineral elements to constitute a comprehensive mineral composition. Both liquid and dry powder mineral element compositions produced by the procedures described herein preferably, but not necessarily, contain a minimum of 8 macro mineral elements and a minimum of 60 micro mineral elements.

Physical testing and analysis was also conducted on the liquid and dry mineral element compositions. Typical specifications of liquid extract solution range in color but preferably are from yellow to amber brown and contain between 1 to 10% by weight of mineral elements, most preferably 3-5%. The solution is acidic with a pH ranging from 2.5-4.5, most preferably from 2.5-3.5. The liquid extract can be dried to produce an anhydrous powder. The anhydrous powder presently ranges in color from light-off-white to brown, but preferably from yellow to golden amber, is insoluble in any non-polar solvent such as hydrophobic liquids (oil and fats), is insoluble in alcohol, and is readily soluble, yet non-swelling, in water and hydro-alcoholic solutions at concentrations of 1 to 5%, most preferably at concentrations of 3-5% by weight. The dry powder is partially soluble or capable of being partially suspended in polar solvent in supersaturated solutions. The dry powder can also be easily suspended in non-polar solvents.

As stated above, both liquid and dry mineral element compositions produced by the procedures described herein will contain a minimum of 8 macro mineral elements and a minimum of 60 micro mineral elements. The micro mineral elements include trace and rare earth mineral elements.

For example, the dry mineral element composition will contain at concentrations ranging from 0.0001-20.00% by weight, most preferably from 0.001% -10% by weight, the macro mineral elements of calcium, chlorine, magnesium, manganese, phosphorous, potassium, silicon, and sodium; and, will preferably contain at least sixty micro mineral elements at concentrations ranging from 0.00001-3.0% by weight, most preferably from 0.0001-1% by weight. The micro mineral elements include aluminum, antimony, arsenic, barium, beryllium, bismuth, boron, bromine, cadmium, cerium, cesium, chromium, cobalt, copper, dysprosium, erbium, europium, fluorine, gadolinium, gold, hafnium, holmium, iodine, indium, iridium, iron, lanthanum, lead, lithium, lutetium, mercury, molybdenum, neodymium, nickel, niobium, palladium, platinum, praseodymium, rhenium, rhodium, rubidium, ruthenium, samarium, scandium, selenium, silver, strontium, sulfur, tantalum, terbium, tellurium, thallium, thorium, thulium, tin, titanium, tungsten, vanadium, ytterbium, yttrium, zinc, and zirconium.

Since the process described herein normally does not introduce any minerals as part of the extraction process, it can be established that any minerals identified and quantified by the process described herein have been captured and recovered from the initial soil matter or the starting raw material. Therefore, it can be established that the original clay or other soil that processed through the extraction method described herein likely include aluminum silicates and other metal silicates in nature which has been naturally enriched with multiple detectable minerals. It can also be established that if a mineral element is identified and quantified in the aqueous liquid extract, it will be identified and quantified in the dry powdered extract in much higher concentrations as a result of drying process or volume reduction.

For example, a lot produced using the soil and extractions methods described herein was tested by independent analytical testing for conducting chemical analysis using standard techniques of identification and quantification for both dry and liquid forms of the comprehensive mineral composition. The results of testing performed at Teledyne Wah Chang Laboratories in Huntsville, Ala., utilizing scientifically accepted and standard equipment such as Titration, Inductively Coupled Plasma, Mass Spectrometry, and Atomic Absorption equipment resulted in the mineral element quantification data set forth below in TABLE I for an aqueous mineral element composition and from the dry mineral element composition that resulted when the aqueous mineral element composition was dried to produce a powder.

TABLE I

| | | |
|---|---|---|
| | Concentration in aqueous | Concentration |
| Element | liquid composition | in dry powder |

Macro Mineral Elements

| | | |
|---|---|---|
| Calcium | 2900 ppm | 8% |
| Chlorine | 170 mg/ml | 0.84%* |
| Magnesium | 460 ppm | 0.95% |
| Manganese | 8.6 ppm | 240 ppm |
| Phosphorous | 0.2 g/L | 0.43% |
| Potassium | 220 mg/L | 1.2% |
| Silicon | 130 mg/L | 0.36% |
| Sodium | 720 mg/L | 2.0% |

Micro Mineral Elements

| Aluminum | 540 ppm | 0.65% |
|---|---|---|
| Antimony | 460 ppb | 16.0 ppm |
| Arsenic | 11 ppm | 3.1 ppm |
| Barium | 340 ppb | 11.0 ppm |
| Beryllium | 0.29 ppm | .01 ppm |
| Bismuth | <50 ppb | <1.00 ppm |
| Boron | 2.0 mg/L | 72.00 ppm |
| Bromine | *Present as part of Chlorine assay | |
| Cadmium | <50 ppb | 1.10 ppm |
| Total Organic Carbon | 12 g/L | Trace |
| Cerium | 1600 ppb | 68.00 ppm |
| Cesium | 82 ppb | 2.00 ppm |
| Chromium | 1.8 ppm | 5.00 ppm |
| Cobalt | 0.25 ppm | 1.00 ppm |
| Copper | 0.09 ppm | <1.00 ppm |
| Dysprosium | 230 ppb | 9.00 ppm |
| Erbium | 150 ppb | 6.00 ppm |
| Europium | <50 ppb | 2.00 ppm |
| Fluorine | *Present as part of Chlorine assay | |
| Gadolinium | 220 ppb | 9.00 ppm |
| Gallium | 70 ppb | 2.40 ppm |
| Germanium | <50 ppb | <1.00 ppm |
| Gold | <50 ppm | <1.00 ppm |
| Hafnium | <0.5 mg/L | 5.00 ppm |
| Holmium | <50 ppb | 2.00 ppm |
| Iodine | *Present as part of Chlorine assay | |
| Indium | <50 ppb | Trace |
| Iridium | <50 ppb | <1.00 ppm |
| Iron | 730 ppm | 1.25% |
| Lanthanum | 650 ppb | 28.00 ppm |
| Lead | <50 ppb | <1.00 ppm |
| Lithium | 0.9 mg/L | <1.00 ppm |
| Lutetium | <50 ppb | <1.00 ppm |
| Mercury | Trace | <1.00 ppm |
| Molybdenum | 3200 ppb | 120.00 ppm |
| Neodymium | 1000 ppb | 45.00 ppm |
| Nickel | 0.74 ppm | 2.00 ppm |
| Niobium | 96 ppb | 3.00 ppm |
| Palladium | <500 ppb | <1.00 ppm |
| Platinum | <50 ppb | <1.00 ppm |
| Praseodymium | 290 ppb | 10.00 ppm |
| Rhenium | <50 ppb | <1.00 ppm |
| Rhodium | <50 ppb | <1.00 ppm |
| Rubidium | 360 ppb | 11.00 ppm |
| Ruthenium | <50 ppb | <1.00 ppm |
| Samarium | 250 ppb | 10.00 ppm |
| Scandium | <400 ppb | 4.00 ppm |
| Selenium | 0.63 mg/L | 21.00 ppm |
| Silver | <0.02 ppm | <5.00 ppm |
| Strontium | 14000 ppb | 420.00 ppm |
| Sulfur | 1.1 g/L | 1.8% |
| Tantalum | <50 ppb | <1.00 ppm |
| Terbium | <50 ppb | 2.00 ppm |
| Tellurium | <50 ppb | <1.00 ppm |
| Thallium | <50 ppb | 1.00 ppm |
| Thorium | 640 ppm | 22.00 ppm |
| Thulium | <50 ppb | 1.00 ppm |
| Tin | <50 ppb | <1.00 ppm |
| Titanium | 9.34 ppm | 210.00 ppm |
| Tungsten | 52 ppb | 17.00 ppm |
| Vanadium | 4.3 ppm | 14.00 ppm |
| Ytterbium | 140 ppb | 6.00 ppm |
| Yttrium | 1300 ppb | 61.00 ppm |
| Zinc | 1.2 ppm | 14.00 ppm |
| Zirconium | 2.0 mg/L | 62.00 ppm |

The mineral element compositions set forth above in Table I were produced from naturally occurring soil the analysis of which is reflected below in Table II.

TABLE II
Analysis of Naturally Occurring Soil
Macro Mineral Elements

| | Concentration in ppm by weight |
|---|---|
| Element | unless noted as % (for weight percent) |
| Silicon | 25.0% |
| Aluminum | 9.3% |
| Potassium | 4.8% |
| Magnesium | .83% |
| Sulfur | 1.6% |
| Iron | 1.6% |
| Calcium | 4.1% |
| Titanium | 0.23% |
| Sodium | 0.138% |
| Manganese | 150 |
| Gallium | 25 |
| Molybdenum | 61 |
| Germanium | 25 |
| Iodine | 7 |
| Bromine | 5.2 |
| Tungsten | 8.1 |
| Hafnium | 2.0 |
| Tantalum | 0.50 |
| Zirconium | 10 |
| Arsenic | 0.2 |
| Antimony | 29 |
| Selenium | 4.1 |
| Zinc | 20 |
| Samarium | 3.5 |
| Holmium | 1.1 |
| Terbium | .62 |
| Iridium | .51 |
| Lutetium | .45 |
| Chromium | 70 |
| Lanthanum | 18 |
| Ruthenium | 7.8 |
| Yttrium | 1.2 |
| Indium | .38 |
| Lead (under) | 17 |
| Niobium | 2.89 |
| Carbon | .19 |
| Hydrogen | .05 |
| Nitrogen | .03 |
| Scandium | 3.7 |
| Cobalt | 4.8 |
| Ytterbium | 1.4 |
| Strontium | 240 |
| Barium | 390 |
| Gold | .68 |
| Europium | .49 |
| Neodymium | 20 |
| Cerium | 40 |
| Cesium | 183 |
| Thorium | Above 100 |
| Uranium | Above 100 |
| Nickel | 60 |
| Beryllium | .10 |
| Bismuth | 14.3 |
| Boron | 7 |
| Cadmium | 1.12 |
| Chloride | 6100 |
| Copper | 2.2 |
| Fluoride | 3.85 |
| Lithium | 1.44 |
| Mercury | 0.166 |
| Palladium | 0.74 |
| Phosphate | 320 |
| Platinum | 0.08 |
| Rhodium | 0.44 |
| Rubidium | 36.5 |
| Silver | 0.3 |
| Tellurium | 0.1 |
| Thulium | 0.65 |
| Tin | 0.44 |
| Vanadium | 8 |
| Dysprosium | 4.0 |
| Praseodymium | 2.0 |
| Thallium | 10 |
| Rhenium | 1.0 |
| Erbium | 2.0 |
| Oxygen | 0.2 |

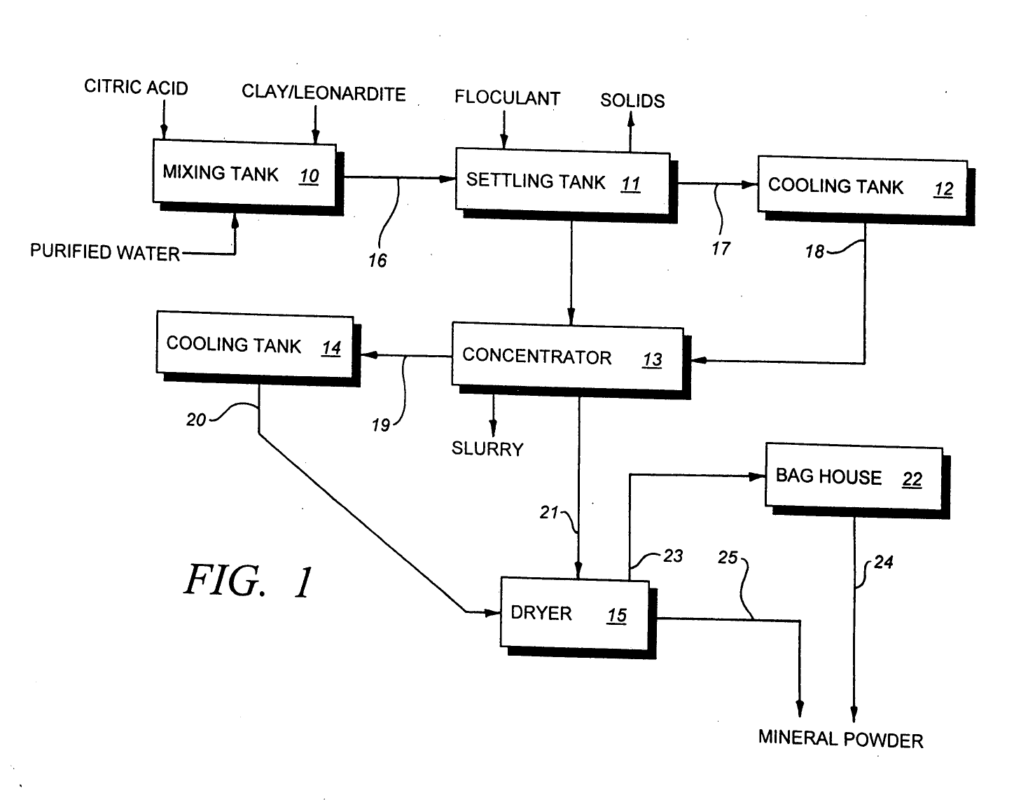

Once a desirable naturally occurring soil or soil combination is obtained, the soil(s) is subjected to the extraction process shown in FIG. 1 and described below in more detail. The selection of an appropriate soil or soil combination is, however, important in the practice of the invention and this process is now described. It is understood that it is possible to incorporate synthetically produced soils or compositions to produce soils used in the invention; however, the use of naturally occurring soils is presently preferred and it is the use of such naturally occurring soils that is now described in detail.

Clay soils, mixtures of clay soils, or mixtures of clay soil(s) and leonardite are presently preferred in the practice of the invention. One reason such soil combinations are preferred is that such soils can be high in the mineral elements deemed important in the practice of the invention. As noted, it is preferred that mineral element compositions produced in accordance with the invention include at least eight macro mineral elements and at least sixty micro mineral elements.

The first step in determining whether a clay soil is acceptable is to determine of arsenic, lead, mercury, and cadmium are each present in acceptably small concentrations. It is presently preferred that the concentration of each of these elements be less than the concentrations shown below in Table III.

TABLE III
Maximum Desired Concentrations of Toxic Elements

| | Maximum Desired Soil Concentration |
|---|---|
| Element | in ppm or ppb |
| Arsenic | 0.2 ppm |
| Lead | 0.17 ppb |
| Mercury | 0.116 ppm |
| Cadmium | 1.12 ppm |

To achieve the desired concentrations noted above, a soil that has a greater than desired concentration of the toxic elements can be admixed with one or more soils containing a lesser than desired concentration of the toxic elements. Further, the maximum desired concentrations of the four toxic elements noted above can vary depending on the intended end use of the mineral element composition produced by the invention. For example, if the mineral element composition is intended to be used in products ingested by human beings, the acceptable levels of the toxic elements normally will be less than if the mineral element composition will be used in agricultural products.

If the soil, or soil combination, has appropriately low concentrations of the four toxic elements arsenic, lead, mercury, and cadmium, the soil is next tested to determine if acceptable concentrations of rare earth elements are present in the soil or soil combination. Desired levels of rare earth elements are set forth below in Table IV.

TABLE IV
Preferred Minimum Concentrations of Selected Rare Earth Elements
in Naturally Occurring Soil

| | Preferred Minimum Soil Concentration |
|---|---|
| Element | in ppm |
| Cerium | 40 |
| Praseodymium | 2 |
| Neodymium | 20 |
| Samarium | 3.5 |
| Europium | .49 |
| Terbium | .62 |
| Dysprosium | 4 |
| Holmium | 1 |
| Erbium | 2 |
| Thulium | .65 |
| Ytterbium | 1.2 |
| Lutetium | .45 |

The concentration of the elements listed in Table IV can vary as desired, but, as noted, it is desirable to have at least the concentration of each element as noted in Table IV. A lanthanum concentration of at least eighteen ppm and a scandium concentration of at least three and seven-tenths ppm are also preferred. Concentrations of promethium and gadolinium are also desirable. In the practice of the invention, at least ten rare earth elements are present in the soil, preferably at least twelve, and more preferably all of the rare earth elements along with lanthanum and scandium. The presence of most or all of the rare earth elements in the soil, and in the mineral element compositions derived from the soil, is believed to be important in improving the efficacy of the mineral element composition when ingested by the body or when transdermally absorbed by the body.

The clay soil or soil combination also includes at least 5% by weight calcium, preferably at least 10% by weight calcium, and most preferably at least 20% by weight calcium. Concentrations of calcium of 25% by weight or greater are acceptable.

The clay soil or soil combination also includes at least 5% by weight silica, preferably at least 10% by weight silica, and most preferably at least 20% by weight silica. Concentrations of silica of 25% by weight or greater are acceptable.

The clay soil or soil combination also includes at least 0.25% by weight phosphorous, preferably at least 1% by weight phosphorous, and most preferably at least 2% by weight phosphorous.

Leonardite is a valuable mineral source in producing soils that are subjected to the extraction process illustrated in FIG. 1.

Once a clay soil or clay soil combination is obtained that contains the requisite mineral elements, the clay soil is subjected to the extraction process of FIG. 1. The following example describes the extraction process by way of illustration, and not limitation of the invention.

FIG. 1 is a block diagram illustrating the extraction process of the invention.

### EXAMPLE 1

### Extraction Process

In FIG. 1, 12,000 pounds of water purified via reverse osmosis or another desired purification process, 200 pounds of citric acid, and 5000 pounds of clay soil are added to the mixing tank 10. The amount of citric acid (or of phosphoric acid or other edible acid(s)) used can be in the range of 0.25% to 7.5% of the weight of water utilized, but typically is in the range of 1.0% to 2.0%. The purified water is produced using any desired water purification technique; however, water purified by reverse osmosis is presently utilized. The water—citric acid—soil slurry is gently agitated (for example, with a blade slowly rotating at from one to ten RPM) for about an hour, although the agitation time can vary as desired. The agitation is preferably non-cavitating and is carried out without forming bubbles in the mixture.

The slurry from tank 10 is directed, as indicated by arrow 16, into a settling tank 11 to permit particulate to settle downwardly out of the slurry. The slurry is maintained in the settling tank 11 for any desired length of time, but this length of time is presently in the range of about one to ten days. As the length of time that the slurry is maintained in the settling tank 11 increases, the amount of liquid that can be drawn out of the tank and sent to cooling tank 12 or concentrator 13 increases and the amount of solids that have settled to the bottom of the tank increases. Chemicals or any other desired method can be utilized to facilitate the settling of solids from slurry directed into tank 10. After the slurry has resided in settling tank 11 for the desired period of time, liquid is drawn out of the tank to cooling tank 12, or directly to the concentrator 13. The solids on the bottom of tank 11 can be directed to tank 10 to be reprocessed, can be discarded, or can be otherwise utilized.

Cooling tank 12 cools the fluid from tank 11 to a temperature in the range of forty to seventy degrees F. (5 to 21 degrees C.). Tank 12 (and 14) is presently cooled with a refrigeration system to cool the fluid in tank 12. Consequently, when fluid contacts the inner cooled wall surfaces of tank 12, the wall surfaces transport heat away from and cool the fluid. Any desired system can be utilized to cool tank 12 (and 14) and/or to cool the fluid in the tank. For example, a coil can be placed in the fluid and cool the fluid without directly cooling the tank walls with a refrigeration or other system. The fluid from tank 11 is cooled to prevent or minimize yeast and mold growth. The fluid in tank 11 normally is heated due to the ambient temperature and not due to any chemical or mechanical action that takes places in tank 11. Cooled liquid from tank 12 is, as indicated by arrow 18, directed from tank 12 to concentrator 13.

The concentrator 13 comprises a thin film composite reverse osmosis system in which fluid is directed into a plurality of long, cylindrical, hollow liquid permeable membrane tubes under pressure; and, in which fluid is forced radially out through the liquid permeable cylindrical membrane wall to increase the concentration of the mineral elements in the fluid. Evaporation is an alternate approach to increasing the concentration of mineral elements in the fluid. A reverse osmosis system is preferable to evaporation because it requires less energy, and because the water that passes radially through the membrane is a source of clean usable water.

One preferred reverse osmosis system includes eight hollow tubes or “vessels” that are about four inches in diameter and forty inches long. Each tube houses three concentric cylindrical membranes. The permeability flow rate is approximately 80% to 95% rejection, depending on the feed rate and the concentration of mineral elements in the fluid being treated. The spacing between the three concentric membranes is about ¼ inch. There are three ring couplers and one end plug per tube. The maximum pressure allowed by the cylindrical membranes is about 600 psig. A pressure of between 300 to 450 is recommended and is normally used. The membranes are to be utilized at a temperature of 135 degrees F. (57 degrees C.) or less. The temperature of the fluid and the membrane is, however, typically maintained in the range of 55 degrees F. to 65 degrees F. (12 to 20 degrees C.). The fluid from tank 11 is processed by passing it sequentially through each of the eight tubes.

If desired, concentration systems other than reverse osmosis systems can be utilized. Such other systems are not believed comparable to a reverse osmosis system in terms of cost and efficiency.

In FIG. 1 the “slurry” by product produced by the concentrator 13 comprises clean usable water with a low concentration of mineral elements. The aqueous concentrate liquid produced by concentrator 13 is, as indicated by arrow 19, directed to cooling tank 14 or directly to dryer 15. Tank 14 cools the concentrate liquid to 40 degrees F. to 70 degrees F. (5 degrees to 20 degrees C.) to prevent the growth and yeast and mold.

The concentrate liquid produced by concentrator 13 has a pH of approximately 3. The concentrate liquid typically includes from three to twelve percent by weight mineral elements, i.e. if the mineral elements are separated from the concentrate liquid, a dry material is produced that has a weight equaling about 3% to 12% by weight of the concentrate liquid. The pH of the concentrate liquid is adjusted by varying the amount of citric acid or other edible acid and/or alkaline or acidic soil added to the mixing tank 10 and is in the range of pH 2.0 to pH 5.0, preferably pH 2.5 to pH 3.5. The pH of the concentrate liquid (and dry powder or other material produced therefrom) preferably is less than pH 4.5. Table I herein illustrates the mineral element present in one concentrate liquid produced by concentrator 13. If necessary, the concentrate liquid is recirculated back through concentrator 13 to increase the mineral element content in the liquid. As the proportion of mineral elements increases, the propensity of mineral elements to precipitate from the concentrate liquid increases. A mineral element concentration of at least eight percent is presently preferred for injection into dryer 15. A mineral element concentration in the range of three to twelve percent or more is beneficial because many prior art processes currently only produce a fluid having a mineral element concentration of about two percent.

Any desired drying system can be utilized. The present drying apparatus consists of a tower into which the concentrate fluid is sprayed. Air in the tower is heated. The concentrate fluid is sprayed in a pattern that causes the spray to swirl down the sides of the tower. As the spray travels down the sides of the tower, the water evaporates, producing powder particles including mineral elements. The powder falls downwardly to the bottom of the tower. Moist air travels upwardly through the center of the tower and is directed 23 to a bag house 22. The moist air enters elongate air-permeable hollow generally cylindrical bags in the bag house. The air travels outwardly through the walls of the bags and leaves behind powder particles on the inside surfaces of the bag. The bags are shaken each thirty seconds to cause the powder on the inner surfaces of the bag to fall downwardly for collection. Table I illustrates the mineral element concentration in the powder produced in dryer 15 when the liquid mineral element concentrate having the composition set forth in Table I was directed into dryer 15. The dry powder mineral element composition of Table I in aqueous solution has a pH of about 3.0.

In one spray system utilized in the dryer 15, the fluid concentrate is directed into dryer 15 under a pressure of about 2500 psi. The orifice size of the spray nozzles utilized is about 0.027 inch. The spray angle of the nozzle is 70 degrees and the average droplet size is about 75 microns.

The use of a nutritional supplement in tablets, soft capsules, bars, processed foods or beverages which contains the small concentrations of the mineral element compositions described herein could be beneficial to health if used to supply sub-toxic dosages of certain mineral elements that can pose a toxic risk. An Acute Oral Toxicity animal study conducted at Northview Pacific Labs in Hercules, Calif. indicated that acute dosages of 1 gram of dry mineral element composition per kilogram of weight of an individual classified the Comprehensive Mineral Composition posed no toxicity risk to an individual. This qualifies the products produced by the processes of the invention as a unique composition that delivers a substantial natural balance of minerals through oral supplementation in a single or multiple dosages for human and veterinary product consumption.

In the area of topical application and delivery of minerals, there is growing evidence that transdermal delivery could be the best route to deliver therapeutic agents, particularly metal drugs. There is also great interest on skin for being the next frontier for better route of delivery of vitamins and minerals for improved systemic absorption and availability. For example, studies at the Graduate School of Science and Technology at Bond University in Australia demonstrated how the gastrointestinal tract presents a significant barrier to the efficient absorption of both orally administered and inject-able dietary essential trace minerals. Their studies indicate that presenting trace minerals which can penetrate the dermis permits their slow release from the skin with more efficient (relative to incipient toxicity) systemic delivery. Examples are given of dermal application of copper, zinc, titanium, platinum and gold complexes to treat chronic inflammatory disease. Some of these compounds are also anti-cancer agents. Other studies have demonstrated that skin penetration of minerals follow a pattern of organ distribution.

The inventors believe that the mineral element compositions described herein can be an ideal multi-mineral product for delivery through the skin qualifying as a unique composition that delivers a substantial natural balance of minerals to the surface of the skin or on stratum corneum for transdermal supplementation. A single or multiple dosage for human and veterinary product application onto the skin would contain small concentrations of the mineral element compositions described herein and could be beneficial to health if used in sub-toxic dosages.

An example of a transdermal product follows.

EXAMPLE 2
Transdermal Mineral Gel Composition

| Ingredients | Weight % |
|---|---|
| Liquid mineral element composition | q.s. to 100% |
| of Table I | |
| Xanthan gum | 0.30 |
| Diethylene glycol monoethyl ether | 12.00 |
| Ethyl oleate | 2.00 |
| Alcohol SDA 40 | 7.00 |

Procedure:
1. Mix xantham gum in liquid mineral element composition using propeller mixer.
2. Add other ingredients one by one.

One Kilogram of Transdermal Mineral Gel Formula Composition will deliver onto the stratum corneum no less than 1 ppm of Macro Minerals consisting of a blend of Calcium, Chlorine, Magnesium, Manganese, Phosphorus, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium, Tellurium, Thallium, Thorium, Thulium, Tin, Titanium, Tungsten, Vanadium, Ytterbium, Yttrium, Zinc, Zirconium, to be absorbed and bioavailable selectively by skin as it is delivered transdermally.

The resulting aqueous solutions from the mineral element composition are highly acidic. Preparation of acidic mineral element solutions can useful, particularly for the personal care industry.

An example of a low pH mineral composition follows:

### EXAMPLE 3

Water—100% as supplied through the process of reverse osmosis.

pH=5.7

### EXAMPLE 4

Water: 95% by weight, as supplied in Example 3 through the process of reverse osmosis.

Dry mineral element composition of Table I: 5% by weight

Mix water and mineral element composition together. The pH of the resulting aqueous solution is 3.0.

The mineral composition aqueous solution of Example 3 is substantially non-irritating to skin and eyes. Acidic solution will normally be irritating to open wounds. For example, aqueous solutions of glycolic acid with a pH=3.0 will sting or burn when applied or upon contact to freshly shaven skin. The pH=3.0 aqueous solution of Example 3 causes little or no sting or burning when applied to freshly shaven skin.

The inventors believe that the mineral compositions described herein can be an ideal multi-mineral product for delivering exfoliation to the skin.

Skin proliferation, the rate at which cell are born at the basal layer and subsequently shed from the body after reaching the upper layers of the stratum corneum, is an important and dynamic function for maintaining healthy skin. For example, psoriatic patients suffer from proliferation disorders as skin cells do not regenerate or desquamate normally. Because minerals such as zinc and copper play a role in skin proliferation, they have been extensively studied for topical application and have been shown to improve certain skin condition disorder. Skin proliferation disorders such as dandruff have also been studied with the use of minerals to bring about improvements.

Altering the rate of skin proliferation has been the mechanism by which many anti-aging skin care products are promoted. As skin ages the skin proliferation rate decreases, and stimulating cell renewal to a rate that is closer to younger skin has proven to improve the general appearance of skin. Ingredient such as retinoic acids, retinol and alpha hydroxy acids (AHA's) are widely promoted on a global scale for their ability to increase cell turnover and promote younger looking skin.

For these reasons AHA's are a commonly added to skin care products including moisturizers, cleanser, toners, and masks. AHA's are naturally derived from fruit and milk sugars and synthetically made as pharmaceutical and cosmetic acidulant ingredient. They are used in skin care as ‘cosmeceutical’ or functional cosmetic ingredients.

The most commonly used AHA's are glycolic acid and lactic acid. AHA's work mainly as an exfoliant of the skin. They cause the cells of the skin to become “unglued” allowing the dead skin cells at the surface of the skin to slough off, making room for re-growth of new skin. They also indirectly stimulate, through the process of irritation, the production of new cells. They have been reported to improve wrinkling, roughness, and pigmentation on skin after long term application and have been extensively studied.

AHA's as used in skin-care products work best at acidic pH's as it is the free acid and not the neutralized or salt counterparts that have been found effective on the skin as exfoliants. Typically, a pH of 3-5 is optimal when utilizing AHA's. As a result, two major side effects of AHA's are irritation and sun sensitivity. Symptoms of irritation include redness, burning, itching, pain, and possibly scarring. There are milder and other forms of exfoliants on the market today than AHA. Beta hydroxy acids such as salicylic acid have been reported to bring about skin cell turnover rate increases. Retinol (the alcohol form of retinoic acid) has also been extensively used.

It was unexpectedly found that the liquid mineral element composition and the dry powder mineral element composition produced in accordance with the invention, as well as solutions of the same, were able to cause skin to exfoliate. There appears to be no prior art suggesting any anticipatory use of minerals as skin exfoliants or to affect cell renewal.

Topical preparations that included the use of the mineral element compositions of the invention were observed to provide multiple skin benefits. Among the benefits observed was mild exfoliation. Exfoliation was subjectively measured by the ability of skin to be renewed after several days of use, with some mild peeling depending on subject. Skin was observed as less sallow and more translucent. Product containing 5% by weight of the dry powdered mineral element composition of Table I in aqueous solution was observed to provide the maximum exfoliation effect.

Typically, AHA products become irritating after several days of use as the skin becomes sensitized to low pH levels of these products. Comparatively, aqueous solutions including 5% by weight of the dry mineral element composition of Table I at a pH of 3 demonstrated the ability to exfoliate skin in a non-irritating manner.

It is therefore novel, at least for the mineral element compositions derived by the extraction process described herein, that the mineral element compositions can serve as a new class of cosmetic and dermatological ingredients of exfoliation with significantly less adverse effects such as burning and irritation.

The use of the comprehensive mineral composition in topical over the counter therapeutic products is believed to be beneficial to skin disorders ranging from severe dry skin to treatment of skin disorders. It is known that many macro and micro mineral elements play important roles in treating skin disorders. For example, copper is essential for production of tyrosinase, an enzyme which is required for the production of melanin for the activation of melanocytes which together with sunscreens protect the skin from UV by initiating tanning. As another example, selenium can help in the treatment and prevention of dandruff and deficiency in the mineral can lead to appearance of premature aging.

It is believed that the comprehensive mineral compositions described herein can be an ideal multi-mineral product for delivery on the skin qualifying as a unique composition that delivers a substantial natural balance of minerals in a single or multiple dosages for human and veterinary product consumption providing mild exfoliation effects.

### EXAMPLE 5

### Exfoliant Cleanser Composition

| Ingredient | Weight % |
|---|---|
| Liquid Mineral Element Composition | q.s. to 100% |
| Of Table I: | |
| TEA Cocoyl Glutamate | 7.00 |
| Glycerin | 5.00 |
| Decyl Glucoside | 5.00 |
| Dimethicone Copolyol Phosphate | 2.00 |
| Preservatives and Fragrance | 1.00 |

Procedure:

Blend each ingredient one at a time to produce final composition. Apply final composition to skin with or without water, gently rub composition into skin for at least 2 minutes, and rinse with water.

One liter of Exfoliant Cleanser Composition delivers onto the stratum corneum no less than one ppm of Macro Minerals consisting of a blend of Calcium, Chlorine, Magnesium, Manganese, Phosphorus, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium, Tellurium, Thallium, Thorium, Thulium, Tin, Titanium, Tungsten, Vanadium, Ytterbium, Yttrium, Zinc, Zirconium

### EXAMPLE 6

### Suspended Minerals Exfoliant Scrub Composition

| Ingredients | Weight % |
|---|---|
| Phase A | |
| Water | q.s to 100% |
| Propylene Glycol | 5.00 |
| Phase B | |
| Hydrogenated Polyisobutene | 10.00 |
| Isopropyl Myristate | 5.00 |
| Mineral Oil | 3.00 |
| PEG 100 Stearate & | 5.00 |
| Glyceryl Monostearate | |
| Polysorbate 20 | 1.00 |
| Beeswax | 2.00 |
| Phase C | |
| Preservatives and Fragrance | 1.00 |
| Dry Mineral Element | 20.00 |
| Composition of Table I | |

Procedure:
1. Blend ingredients listed above under Phase A and heat to 75 C.
2. Blend ingredients listed above under Phase B and heat to 77 C.
3. Add Phase B to Phase A at 77 degrees C. and blend with propeller mixer to produce intermediate composition.
4. Cool intermediate composition to 40 C, and add ingredients listed above under Phase C to intermediate composition one ingredient at a time to produce final composition.
5. Cool final composition to 25 C. Apply final composition to skin with or without water, gently rub composition into skin for at least one minute, and rinse with water to exfoliate skin.

One liter of Suspended Minerals Exfoliant Scrub Composition delivers onto the stratum corneum no less than one ppm of Macro Minerals consisting of a blend of Calcium, Chlorine, Magnesium, Manganese, Phosphorus, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium,. Tellurium, Thallium, Thorium, Thulium, Tin, Titanium, Tungsten, Vanadium, Ytterbium, Yttrium, Zinc, Zirconium.

### EXAMPLE 7

### Antibacterial Exfoliant Toner Composition

| Ingredient | Weight Percent |
|---|---|
| Liquid Mineral Element | q.s. to 100% |
| Composition of Table I | |
| Hydroxyethyl cellulose | 1.00 |
| Ethyl Alcohol | 62.00 |

Procedure:

Blend ingredient together one at a time at room temperature to produce the exfoliant toner formula composition. Apply the resulting composition by gently rubbing a small amount into the dermis for about thirty seconds. Then rinse dermis to remove any remaining composition.

One liter of Antibacterial Exfoliant Toner Composition will deliver onto the stratum corneum no less than one ppm of Macro Minerals consisting of a blend of Calcium, Chlorine, Magnesium, Manganese, Phosphorus, Potassium, Silicon, Sodium, and no less that 0.0001 ppm of Micro Minerals consisting of a blend of Aluminum, Antimony, Arsenic, Barium, Beryllium, Bismuth, Boron, Bromine, Cadmium, Cerium, Cesium, Chromium, Cobalt, Copper, Dysprosium, Erbium, Europium, Fluorine, Gadolinium, Gold, Hafnium, Holmium, Iodine, Indium, Iridium, Iron, Lanthanum, Lead, Lithium, Lutetium, Mercury, Molybdenum, Neodymium, Nickel, Niobium, Palladium, Platinum, Praseodymium, Rhenium, Rhodium, Rubidium, Ruthenium, Samarium, Scandium, Selenium, Silver, Strontium, Sulfur, Tantalum, Terbium, Tellurium, Thallium, Thorium, Thulium, Tin, Titanium, Tungsten, Vanadium, Ytterbium, Yttrium, Zinc, Zirconium.

Skin is the largest organ of the body. As we age, skin, like many other organs, skin gradually loses its ability to function as it once did. It becomes less efficient in all the processes it possesses as we age, and so does its ability to regenerate and repair itself.

One important skin function that diminishes is based on a reptilian similarity human skin possesses and that is its constant state of renewal. As a result, it is known to those skilled in the field that as skin ages, there is an increase in surface accumulation of dead cells as cell renewal rates decrease.

Skin cell regeneration, a process of replenishing older cells with fresh cell in the constant cycle of renewal, is important in maintaining viable cell health and a healthy youthful appearance. New skin cells are constantly born at deepest basal layer underneath the visible layer by a process of continual cell (keritinocytes) division. As new skin cells mature and nourish the underlying tissue, they proliferate until reaching the visible surface of the skin, eventually losing their nucleus, becoming flattened, and dying while being shed from the body as other new vibrant cells replace them from underneath in a harmonious sequence. The process is often referred to as skin cell renewal, cell turnover, cell proliferation, or cell regeneration.

As we age, several possible events trigger a decrease in the rate of cell regeneration. Among them are hormonal and other physiological changes that tire the renewal process. It is also possible that weakened blood vessels within the skin and reduced cell surface area limit the nutrient uptake of cells and this slows renewal. It is further possible that the natural cohesion factors that function to release and shed skin loss their efficiency for releasing skin at a sustain pace with the rebirth of new cells, allowing dead skin cells to simply accumulate on the surface.

When cell renewal slows down the visible result of excess accumulation of dead skin cells creates a dull, dry, thicker layer of skin. The excess accumulation of surface skin cells works against the natural glow of fresher cells and accentuates gray skin tones as well as accentuates minor skin imperfections, skin dryness, and accentuation of wrinkles.

Skin exfoliants, representing topically applied products that remove or accelerate the removal of surface skin cells, are commonly used to accelerate the process of surface skin cell renewal. Exfoliant products are known to exist in both chemical and abrasive forms. Dermatological exfoliants, are those representing products and devices used in professional offices of dermatologist and estheticians utilizing chemical and abrasive forms intended to be left on the skin for limited time before removal. Over-the-counter and retail cosmetic chemical exfoliants, which may come in finished formulations such as gels, creams or lotions, can be left the skin overnight or removed shortly after application. Most, if not all liquid abrasive consisting of suspended particulate exfoliant compositions (i.e. scrub cleansers) are intended for rinse off after use.

Examples of abrasive exfoliant compositions have been cited extensively in patent art. For example, U.S. Pat. No. 6,432,430 teaches of an exfoliating scrub composition of walnut shells which is removed after application. U.S. Pat. No. 6,294,179 teaches a method of exfoliating skin using abrasive particles of a mean diameter of 40-400 micrometers and a bulk density of 1 to 4. U.S. Pat. No. 6,764,991 teach an exfoliant toilet bar composition which utilizes physical exfoliant particles. U.S. Pat. No. 3,092,111 cites mineral particles (such as Aluminum Oxide, bentonite, diatomaceous earth) as abrasive in exfoliant paste compositions.

Many examples of chemical exfoliant compositions can be cited in the patent art using alpha hydroxyl acids, and even a combination of chemical and physical abrasive exfoliant composition can be exemplified in U.S. Pat. No. 5,939,085.

Most, if not all, physical exfoliant and powdered cosmetic compositions are not intended to be applied to the face just prior to retiring for the night because such compositions transfer from the face to a pillow or other bedding. Consequently, women normally sleep without makeup applied to their face.

Nighttime may be an advantageous time for an individual to apply exfoliant and other compositions to the face, or other epidermal areas, because the individual is not engaged in other activities and because when the body is resting it sometimes is better able advantageously to react to or to utilize certain compositions.

The invention describe herein relates to a leave-on exfoliant powder composition that is used overnight during sleep or resting hours, or is used during the day. Furthermore, the invention described herein relates to a leave-on powdered exfoliant pigmented product that is used during daytimes or during sleep or resting hours.

An improved exfoliant composition has been discovered that can be facially applied just prior to bedtime and that generally remains on or with the user's skin during sleep and, therefore, does not transfer to bedding; or, does not transfer to clothing when worn during the day. The exfoliant is also minimally irritating to the skin.

The improved exfoliant composition is in the form of a powder. Although it is possible for the power to have minimal amounts of water of hydration or other moisture, for example less than 5% by weight water, the powder preferably contains no water. The size of particles in the powder is in the range of 45 to 300 microns, preferably 50 to 200 microns. Since the powder composition contains little to no water, a relative pH can not be measured. However, a 10% aqueous dispersion of the powder composition in water provides a pH range of 5.0 to 7.0, preferably 5.5-6.5.

The powder is hygroscopic, which has a high affinity to moisture and even in dry form after application to the face, is capable of binding to moisture from the skin or from the atmosphere. This hygroscopic character is seen mostly upon application to the skin where it is freely exposed to skin and environmental moisture. Once the powder is applied to skin, the powder adsorbs moisture from the skin and the ambient air. This hygroscopic effect is capable of facilitating the manner in which other ingredients can be hydrolyzed and be utilized by skin. For example, utilization of Vitamin C and other water soluble vitamins in the powder compositions can be more readily bio-available for utilization by the skin once they are hydrolyzed by the hygroscopic nature of the powder composition.

Hygroscopic powder provides the dry exfoliating composition with a multiple-function action that progresses through multiple stages. In the first stage, when the powder is initially applied to the skin, it is dry and may, because of the dry particulate, may work primarily as a gentle abrasive. In the second stage, as the composition adsorbs moisture from skin or environmental air, and the moisture can interact with vitamins and other components in the composition to initiate or support enzymatic or other mechanisms that facilitate exfoliation and facilitate moisturizing the skin. One quantity of moisture may be necessary to solubilize a particular vitamin or mineral to facilitate its absorption by the skin. Another, greater, quantity of moisture may have to be adsorbed to solubilize another vitamin or to moisturize noticeably the skin.

One advantage of the dry exfoliating fine powder composition of the invention is that is it relatively stable because it is in a dry state, which provides an extended shelf life. In addition, as noted, the fine powder blends into the skin, and ordinarily does not transfer to bedding while the user sleeps. Since the powder blends into and remain on the skin, colorants and other compounds found in cosmetics can be incorporated in the powder, which permits the powder to function both as cosmetic for beautifying appearance and an exfoliant.

When the dry exfoliating composition of the invention is applied daily to skin in layers of one to three, the composition may remove dead skin at a rate comparable to milder conventional exfoliation products such as products containing a low concentration of Alpha Hydroxy Acids.

In order to produce a reasonable amount of skin exfoliation, the dry exfoliating composition of the invention is applied at least once a day during evening in one to three layers for at least 30 consecutive days, preferably 60 consecutive days, and most preferably 90 consecutive days. During any such consecutive day period, missing one or two days ordinarily will not significantly adversely affect the skin exfoliation accomplished by utilizing the dry exfoliating composition of the invention. Consequently, as used herein, it is understood that the terminology consecutive day period indicates that during one or two days in such period the exfoliating composition may not be applied.

In certain cases, uses of specific vitamins may be advantageous within the composition. In the case of Vitamin C, the anhydrous powder composition is particularly advantageous as Vitamin C is a highly unstable compound when in solution. Many references regarding the poor stabilize of Vitamin C can be cited, but as an example of a patent reference which clearly elude to the formulation challenge when using Vitamin C, U.S. Pat. No. 5,935,584 teaches a manner in which to keep the Vitamin C in separate compartment as a dry powder as part of dual system package to preserve the stability until combined with the water containing vehicle of a separate compartment upon application.

Surprisingly, the powder composition discovered and described herein is capable of exfoliating skin. Even though the composition of the invention comprises a dry powder, the composition apparently only minimally irritates the skin of a user while exfoliating on a par with many conventional exfoliating.

One important component of the improved dry powder exfoliant composition is a hygroscopic powder comprised of minerals. The mineral powder preferably includes both macro and micro minerals; preferably includes at least eight macro minerals (calcium, chlorine, magnesium, manganese, phosphorus, potassium, silicon, sodium) and sixty micro minerals; preferably has low concentrations of arsenic, lead, mercury and cadmium; and, preferably includes rare earth elements. One desirable, apparently important, component of the mineral powder is a hygroscopic powder mineral composition of the type set forth in Table I herein, preferably is derived from naturally occurring clay soil. The hygroscopic powder mineral composition can comprise from 0.01 to 100% by weight, preferably from 0.01 to 10% by weight, and most preferably 0.01 to 5.0% by weight of the powder exfoliant composition of the invention. The hygroscopic powder mineral composition of Table I adsorbs moisture, i.e., attracts and holds moisture to the surface of the powder particles. Although the powder composition of Table I can be utilized alone, combining it with other minerals, vitamins, herbs, etc. can improve the effectiveness of the composition as a dry skin exfoliant.

Examples of vitamins that are available in powder form and that can be utilized in the powder exfoliant composition of the invention include Vitamin A acetate, Vitamin A palmitate, thiamine mononitrate (Vitamin B1), Riboflavin (Vitamin B2); Niacinamide (Vitamin B3); Calcium d Panthothenate (Vitamin B5); Pyrixidine Hydrochloride (Vitamin B6); Cyanocobalaimin (Vitamin B12); Ascorbic Acid (Vitamin C) and salts like sodium or calcium ascorbate; Beta Carotene; Vitamin D2 (Erogcalciferol); Vitamin D3; Vitamin E Acetate (Tocopheral acetate); and, Vitamin K. Vitamins in powder form can comprise from 0.01 to 100% by weight, preferably from 0.01 to 10% by weight, and most preferably 0.01 to 5.0% by weight of the powder exfoliant composition of the invention. A vitamin (or the mineral composition of Table I) in powder form can facilitate the removal of dead skin cells while providing a nutrient value that maintains or improves the health of underlying living skin, or, that helps repair any damage to underlying living skin cells.

The ascorbic acid (Vitamin C) can, if available in powder form, comprise an ester of ascorbic acid selected from the group consisting of fatty acid monoesters, fatty acid diesters, fatty acid trimesters, fatty acid tetraesters, and mixtures thereof.

The ascorbic acid can, if available in powder form, comprise an ester of ascorbic acid selected from the group consisting of ascorbyl palmitate, ascrobyl laurate, ascorbyl myristate, ascrobyl stearate, ascorbyl diplamitate, ascorbyl dilauarte, ascorbyl dimyristate, ascorbyl distearate, ascorbyl tripalmiate, ascorbyl trilaurate, ascorbyl trimyristate, ascorbyl tristearate, ascorbyl tetrapalmitate, ascorbyl tetralaurate, ascorbyl tetramyristate, ascorbyl tetrastearte, and mixtures thereof.

The ascorbic acid can, if available in powder form, comprise a salt of ascorbic acid selected from the group consisting of ascorbyl phosphate, ascorbyl sulfate, and mixtures thereof.

The ascorbic acid can, if available in powder form, comprise a salt of ascorbic acid selected from the group consisting of L-ascorbic acid 3-phosphate, L-ascorbic acid 2-phosphate, L-ascorbic acid 3-pyrophosphate, bis (L-ascorbic acid 3,3-) phosphate, L-ascorbic acid 3-sulfate, L-ascorbic acid 2-sultate, L-ascorbic acid 3-pyrosulfate, bis (L-ascorbic acid 3,3-) sulfate, and mixture thereof.

In one embodiment of the powder exfoliant composition of the invention, the ascorbic acid can comprise from 0.01% to 100% by weight of the powder exfoliant composition.

An additional component of the powder exfoliant composition of the invention can consist of plant derived powdered ingredients that have been lyophilized or otherwise rendered dried. Such plant ingredients can include Aloe Barbadensis as in Aloe Vera Powdered Juice Extract, Camellia Sinensis as in Green or White Tea Powdered Extract, Rosemarinus Officinale as in Rosemary Powdered Extract, Phyllanthus Emblica as in Emblica Fruit Extract, and Triticum Vulgare as in Whet Germ Extract. The plant derived powdered extracts provide antioxidants and natural anti-inflammatory effects to the composition and their content in the powder exfoliant composition can range from 0.001% to 5% by weight, preferably from 0.001 to 1% by weight, and most preferably from 0.005% go 0.5% by weight.

Since the powdered composition has as one objective beautifying the appearance of skin during the evening resting or sleeping hours, the powder exfoliant composition can include ingredients used in color cosmetics. Such ingredients include mineral-derived powders that provide absorbency, coverage, sheen, opacity, and color. Examples of such powders are Bismuth Oxychloride, Mica, Titanium Dioxide, Iron Oxides, Zinc Stearate, Barium Sulfate, Aluminum Starch Oxtenyl Succinate, and Silica. Such cosmetic powers can, in the powder exfoliant composition, comprise 0.01% to 50% by weight, preferably 0.01% to 40% by weight, and most preferably 0.01% to 25% by weight.

The powdered exfoliant composition can be applied as desired, but typically a powder puff or other applicator is utilized to apply once daily one to three layers of the powder composition to the skin, after which the powder composition is gently massaged into the skin.

### EXAMPLE 8

### Powder Exfoliant Composition

The following ingredients are provided.

| Ingredient | Weight % |
|---|---|
| Bismuth Oxychloride (opacifiers/sheen) | 26.0 |
| Mica/Iron Oxide and Barium Sulfate (sheen/pigments) | 20.6 |
| Aluminum Strach Octenyl Succinate (binding agent) | 20.0 |
| Mica, Iron Oxide, and Titanium Dioxide (sheen/pigments) | 10.0 |
| Illite (clay) | 8.0 |
| Zinc Stearate (binding agent) | 4.0 |
| Silica (smoothing agent) | 3.0 |
| Soil Mineral Concentrate (powder) of Table I (exfoliant) | 2.5 |
| Ascorbic Acid (micro powder) (exfoliant; antioxidant) | 5.0 |
| Aloe Barbadensis Juice (powder form)(emollient) | 0.10 |
| Phyllanthus Emblica Fruit Extract (antioxidant; | 0.50 |
| anti-inflammatory) | |
| Dry Vitamin A Palmitate (exfoliant; antioxidant) | 0.05 |
| Tocopheryl Acetate (antioxidant) | 0.05 |
| Ectoin (antioxidant) | 0.05 |
| Cyclopia Intermedia (antioxidant) | 0.05 |
| Triticum Vulgare (Wheat) Germ Extract (antioxidant) | 0.05 |
| Rosmarinus Officialis (antioxidant) | 0.02 |
| Camellia Sinensis (antioxidant) | 0.02 |
| Aspalathus Linearis (antioxidant) | 0.01 |
| TOTAL | 100.00 |

Procedure:

Admix all ingredients in a blender and pulverize to powder comprised of particles each having a maximum width in the range of 100 to 200 microns. Package powder in sealed container to prevent powder from adsorbing moisture from the air.

One preferred embodiment of the invention simply comprises one or more exfoliant powders.

Another preferred embodiment of the invention simply comprises one or more pigments and one or more exfoliant powders.

A further preferred embodiment of the invention comprises pigment(s), exfoliant(s), and antioxidant(s).

Clay(s), a smoothing agent(s), an emollient(s), and/or an anti-inflammatory can also be included in the preferred embodiments of the invention to enhance the exfoliation and health of the skin.

As used herein, a therapeutically effective amount of an embodiment of the dry powder exfoliant composition of the invention is an amount sufficient to enhance and improve the normal rate of exfoliation of the skin of an individual.

### EXAMPLE 9

The powder of Example 8 was clinically tested at Clinical Research Labs (CRL) in New Jersey with a 200 woman panel test for Repeat Insult Patch Testing (RIPT). Unlike most abrasive exfoliating products and chemical (AHA) products which may show irritation scores, the powder of Example 8 showed “0” scores across the entire study. This qualifies the powder of Example 8 as a non-irritating, exfoliation product. Although not yet clinically demonstrated, one reason the powder is believed to be non-irritating is the ability of the powder to deliver mineral nutrients to living cells in the skin and enzymatically causing exfoliation, thus providing a non-irritating pathway relative to conventional chemical or physical exfoliants.

### EXAMPLE 10

A study was performed using the powder of Example 8. Thirty female subjects ranging in age from 30 to 46 years old were selected. The subjects were in general good health; refrained from taking any systemic medications during the study; refrained from use of suntan salons and excessive exposure to the sun during the study; used a normal wash routine during the study but refrained from use of washcloths, loofahs or coarse sponges on the test sites on the subjects' skin; were not pregnant or lactating and agreed to be abstinent or practice a medically acceptable form of contraception during the study; had not participated in a similar study within two weeks of the initiation of the study; and, did not have know allergies to the ingredients of the powder of Example 8.

The subjects were each given a bar of non-moisturizing soap (Aveeno™ Balancing Bar) to use for a 7-day pre-conditioning phase. Subjects were instructed to discontinue use of moisturizers, lotions, sunscreens, skin treatment products, cleansers, body scrubs, washcloths, loofahs and/or coarse sponges on the arms for the entire length of the study.

After the 7 day pre-conditioning phase was completed, three sites were identified on each subject. Two test sites (measuring 5 cm by 5 cm) were selected on each volar surface of the right and the left forearm between the elbow and the wrist, and each site was assigned 1 of 2 test material treatments. The first treatment included the powder of Example 8 alone. The second treatment included the powder of Example 8, followed by one (1) application of Cetaphil™ Daily Facial Moisturizer with SPF15. Assignment of treatment to the right or the left forearm was determined by a computer-generated randomization code. Treatment designation remained consistent throughout the study period. For each subject, one control site was selected on either the right or the left bicep, as specified by the computer-generated randomization code. The control site remained untreated for the duration of the study.

The test and control sites of each subject were cleansed with 70% isopropyl alcohol, and treated sites were outlined with gentian violet marking pen in order to assist subjects in the location of appropriate test material application sites. The centers of the test and control sites were patched under occlusive conditions (Coverlet™ Adhesive Dressing, Beiersdorf Laboratories Inc., Norwalk, Conn.) with approximately 0.2 grams of Dansyl Chloride (5% in Petrolatum). After being patched with Dansyl Chloride, subjects were instructed to leave the patches in place for 24 hours from the time of application. Approximately 24 hours post-patching, subject returned to CRL to have the Dansyl Chloride patches removed. At this time, the test and control sites (treat and untreated) were examined use a long wavelength UV lam (Wood's Lamp, Spectroline ENF-260C, Spectronics Corporation, Westbury, N.Y.). Examination of the forearms was conducted in a darkened room.

Each site was graded for the presence of fluorescence by an evaluator that was blinded as to the treatment assignment for each site. Fluorescense was graded according to the following scale (half-point scoring was acceptable):

5—Uniform, very bright

4—Uniform, moderate brightness

3—Faded, yet visible fluorescence

2—Faded, spotty appearance

1—Complete disappearance

The day following the removal of the Dansyl Chloride patch and grading of test and control sites, subjects were given the test material and a Daily Diary, in which to record each application and any comments related to the test material use. Each subject was directed to perform specified product applications on the designated forearm site twice daily, 7 days per week. All subjects returned to Clinical Research Laboratories, Inc. daily (Monday through Friday only) for approximately three consecutive weeks. Each subject was permitted one missed visit throughout the study period. Missed visits and weekend days were assigned scores equal to the average of scores for the preceding evaluation and the evaluation following the missed visit/weekend day. Each weekend day was assigned the same average score. All subjects were instructed to return the Daily Diary as well as any remaining test product at the final visit. Fluorescence scores at each visit were recorded for all sites.

The test material application instructions consisted of the following procedures:

Treatment 1
Powder of Example 8 was applied by using a powder puff to swipe the forearm skin twice with the powder of Example 8 and then the powder was rubbed into the skin uniformly, until fully absorbed.

Treatment 2
Powder of Example 8 was applied by using a powder puff to swipe the forearm skin twice with the powder of Example 8, and then the powder rubbed into the skin uniformly, until fully absorbed. One pump of Cetphil™ Daily Moisturizer with SPF 15 was then applied and rubbed into the skin until completely absorbed.

Twenty-five subjects completed the study. Five subjects discontinued study participation for reasons unrelated to the test material.

Cumulative fluorescence scores for treated and control sites on the subjects' arms we calculated at Days 3, 5, 7, and 23.

The cumulative fluorescence scores at Day 3, Day 5, and Day 7 evidenced statistically significant differences in measures of the rate of exfoliation between Treatment 2 (powder of Example 8 with Cetphil™ Daily Moisturizer with SPR 15) and the control, demonstrating a faster rate of epidermal cell exfoliation for sites treated with Treatment 2 relative to that of untreated control sites. For cumulative fluorescence scores at Day 3, Day 5, and Day 7, there were no significant differences between Treatment 1 (powder of Example 8, used alone) and the control.

The cumulative fluorescence scores at Day 3, Day 5, and Day 7 evidenced statistically significant differences in measures of the rate of exfoliation between Treatments 1 and 2 and the control, demonstrating a faster rate of epidermal cell exfoliation for sites treated with Treatments 1 and 2 relative to that of untreated control sites. Each site treated was treated only with Treatment 1 or only with Treatment 2.

### EXAMPLE 11

An independent controlled laboratory clinical study was performed utilizing the composition of Example 8. Of the 36 women that completed the study, over 67% of the women felt that the composition of Example 8 improved their skin by (1) improving skin texture, (2) producing smoother skin, (3) refreshing the skin, (4) revitalizing the skin, (5) producing firmer more resilient skin, (6) permitting the skin to retain more moisture, (7) producing clearer skin, (8) producing skin with fewer imperfections, (9) producing a more even skin tone, (10) producing less visible pores, (11), producing skin that feels healthier, and (12) feeling good when applied to the skin.

### EXAMPLE 12

### Exfoliant Powder Composition

The following ingredients are provided.

| Ingredient | Weight % |
|---|---|
| Ascorbic Acid (micro Powder) | 10.0 |
| Mineral Composition of Table I (Powder) | 90.0 |
| TOTAL | 100.00 |

Procedure:

All ingredients are mixed together in a blender and pulverized to powder comprised of particles each having a maximum width in the range of 100 to 200 microns (i.e., micrometers). Powder is packaged in sealed container to prevent powder from adsorbing moisture from the air. The powder is hygroscopic. The pH of an aqueous dispersion containing five percent by weight of the mineral powder in this Example 12 typically is about 3.0, and can be in the range of 3.0 to 6.5.

### EXAMPLE 13

A study is performed utilizing the powder composition of Example 12. The Powder of Example 12 is applied by using a powder puff to swipe the forearm skin twice and then the product is rubbed into the skin uniformly, until fully absorbed. Of the women completing the study, over 70% felt that the composition of Example 12 improved their skin by (1) improving skin texture, (2) producing smoother skin, (3) refreshing the skin, (4) revitalizing the skin, (5) producing firmer more resilient skin, (6) permitting the skin to retain more moisture, (7) producing clearer skin, (8) producing skin with fewer imperfections, (9) producing a more even skin tone, (10) producing less visible pores, (11), producing skin that feels healthier, and (12) feeling good when applied to the skin. In some cases, the composition is applied just prior to bedtime. The composition does not, while the subject is sleeping, transfer from the skin of the subject to bedding in any manner visually noticeable to the naked eye. The nightclothes worn by each subject do not cover the forearm so that the forearm directly contacts bedding while the subject sleeps. Each of the women experiences minimal or no skin irritation when the powder was applied.

### EXAMPLE 14

### Exfoliant Powder Composition

The following ingredients are provided.

| Ingredient | Weight % |
|---|---|
| Mineral Composition of Table I (Powder) | 100.00 |
| TOTAL | 100.00 |

Procedure:

Powder is pulverized to powder comprised of particles each having a maximum width in the range of 100 to 200 microns. Powder is packaged in sealed container to prevent powder from adsorbing moisture from the air. The powder is hygroscopic. The pH of an aqueous dispersion containing five percent by weight of the mineral powder in this Example 7 typically is about 3.0, and can be in the range of 3.0 to 6.5.

### EXAMPLE 15

A study is performed utilizing the powder composition of Example 14. The Powder of Example 14 is applied by using a powder puff to swipe the forearm skin twice and then the product is rubbed into the skin uniformly, until fully absorbed. Of the women completing the study, over 70% felt that the composition of Example 12 improved their skin by (1) improving skin texture, (2) producing smoother skin, (3) refreshing the skin, (4) revitalizing the skin, (5) producing firmer more resilient skin, (6) permitting the skin to retain more moisture, (7) producing clearer skin, (8) producing skin with fewer imperfections, (9) producing a more even skin tone, (10) producing less visible pores, (11), producing skin that feels healthier, and (12) feeling good when applied to the skin. In some cases, the composition is applied just prior to bedtime. The composition does not, while the subject is sleeping, transfer from the skin of the subject to bedding in any manner visually noticeable to the naked eye. The nightclothes worn by each subject do not cover the forearm so that the forearm directly contacts bedding while the subject sleeps. Each of the women experiences minimal or no skin irritation when the powder was applied.

### EXAMPLE 16

Examples 12 and 13 are repeated utilizing a powder form of Vitamin A in place of the Vitamin C powder. Similar results are obtained.

### EXAMPLE 17

Examples 12 and 13 are repeated utilizing a powder form of Vitamin B12 in place of the Vitamin C powder. Similar results are obtained.

### EXAMPLE 18

Examples 12 and 13 are repeated utilizing, in place of the Vitamin C powder, a powder containing equal parts of powder forms of Vitamin B2, Vitamin E acetate, and Vitamin K. Similar results are obtained.

### EXAMPLE 19

Examples 8 to 20 are repeated except that the powders utilized are comprised of particles having a width each in the range of about 75 to 275 microns instead of in the range of 100 to 200 microns. Similar results are obtained.

### EXAMPLE 20

Example 10 is repeated except that the powder is not applied to the arm of each subject. Instead two test sites and a control site on the face of each subject are selected. Similar results are obtained.

### EXAMPLE 21

Example 20 is repeated except that the powder exfoliating composition is applied facially by each individual just prior to bedtime and the composition does not, while the individual is sleeping for an eight hour period, transfer from the skin of the individual to pillows or other bedding in any manner visually noticeable to the naked eye. When a powder exfoliating composition of the invention is applied to skin of an individual that is exposed to and contacts a pillow or other bedding while the individual is sleeping, then less than 80%, preferably less than 10%, more preferably less than 5%, and most preferably less than 2% by weight of the composition transfers from the skin of the subject to the bedding while the individual is sleeping for an eight hour period. The hygroscopic property of the powder composition, as well as the non-existent or low water content of the powder composition and the fine particle size of the composition, are believed responsible at least in part for the non-transference of the powder from the skin of an individual to bedding. It has been well established that oxidation or “oxidative stress” to living cells is one of the major destructive factors that works against the integrity of healthy tissue. Oxidation has also been implicated in accelerating aging and disease.

Oxidation occurs as a result of both intrinsic and extrinsic factors which initiate the formation and propagation of free radicals. Free radicals are reactive molecules which slowly attack healthy cells by targeting cell membranes and subsequently affecting normal function and structure in a negative manner. Oxidation by its own mechanism is a self propagating event that cascades to further oxidation until enough damage is done to alter DNA, and or permanently destroy healthy cells.

Antioxidants are molecules or compounds which have a direct effect on free radicals by quenching or blocking the highly reactive molecule and preventing it from cascading and causing continued damage to cells.

Intrinsic oxidation factors are the result of normal metabolic function, as for example stemming from digestion of proteins, while others are chemically triggered as exemplified by free radicals generated as a result of high levels of stress. Extrinsic factors can for example range from pollution, smoking, and UV exposure.

A healthy diet of fruits and vegetables delivers natural antioxidants that suppress many of the oxidative reactions triggered by intrinsic factors. However, with the increase in extrinsic factors such as pollution and UV rays, oral supplements rich in antioxidants have become essential to fight against the ravishes of free radicals. Antioxidant supplements consist of vitamins such as vitamin E (tocopherol), minerals such as zinc and selenium, and fruit and vegetables extract rich in polyphenols.

In skin, free radicals and the important role of antioxidant in personal care products is well known to those who practice in the art. Oxidation or free radical generation can accelerate all the factor of aging and even promote skin cancer. Hence, the skin care industry practices the use of antioxidant in many formulations as a way to help skin fight against free radicals.

Unfortunately certain skin care products themselves have been known to create generation of free radicals. Among these potentially oxidizing product are skin cleansers since their propensity to irritation and inflammation is significant and both of these are interrelated with free radical formation.

The need for cleansing of skin dates back to the beginning of humankind. Use of water was the most practical means to free skin from dirt and impurities, and for reducing body malodors. It also probably represents the most gentle and least oxidizing form of cleansing.

Early forms of soap, a combination of fats, oils and salt, subsequently became popular during ancient Roman times as a method of removing bodily dirt and grime through its detergent action when combined with water. Later, goat fat, water and potassium carbonate ash created a more solid form of soap which was more practical for use in bathing. As greater advances in soap making techniques and industrialization came about, solid soap became the most popular cleansing aid during the mid 19^{th} century. As soap evolved to modern sophistication, so did the incidence for irritation and inflammation and as a result the greater the propensity for generation of free radicals.

Facial cleansing is perhaps to the most critical form of skin cleansing from a sensitivity standpoint. Facial skin, and in particular the area surrounding and including the eyes, are highly susceptible to irritation caused by soap. In addition, facial skin seems to be more prone to sensitivity as it relates to allergies, erythematic reactions such as redness, inflammation, and dryness as it relates to cleansing products used. Because facial cleansing is also perhaps the most obvious visible site of irritation relative to other parts of the body, this also plays a role in how consumers perceive gentleness during cleaning, creating psychological sensitivity to any facial cleansing product as it relates to hygiene and beautification in a safe manner.

Facial cleansing, particularly with women, who have increased their use of cosmetics around the world throughout the last century, has become a more challengeable task for soap, as greater cleansing action requires greater use of soap, particularly at the end of the day when make-up removable is typically performed. Unfortunately, soap is not considered mild enough for facial cleansing as it can be irritating and drying, mainly due to the harshness of the nature of the surfactants, and to the pH which tends to be much above pH 7, a pH not ideally compatible with skin natural pH of 5.2-5.8. Furthermore, the higher pH of soap could be conducive to bacterial growth making it a risk to acne prone users on facial area.

Thus emollient cream cleansers, also referred to as cold creams, became very popular after the turn of the 20^{th} century as a way to replace soap for removing dirt, grime and make-up without the irritating or drying effect on the skin.

Today's facial cleansing products have evolved to provide effective cleansing attributes with skin conditioning benefits with greatly reduced incidences of irritation. Cleansers can be found in many forms ranging from solids natural systems such as glycerin soaps, to water based single phase mild surfactant foaming products, to more emollient dual phase emulsion cleansers that can be effective in cleansing facial skin with very little foaming effect during application and rinsing. Product forms can range from liquid, liquid gels, lotions, creams, mousses, and towelletes as examples.

The invention described herein pertains to a method of producing a mineral composition described herein, and to the discovery that such mineral composition has an usually strong antioxidant effect when compared to those seen in the most popular antioxidant renowned fruits.

The antioxidant properties displayed by the mineral composition can be either hydrophilic in its' antioxidant action and specific to quenching free oxygen radicals generated, and/or lipophilic in its' antioxidant action and specific to lipid peroxidation as they effect hydrophobic cell membranes or interstitial lipid structure which makes up the cohesive and barrier function of the skin.

More specifically, a determination of oxygen radical absorbance capacity (ORAC) was conducted at Sunny Biodiscovery Laboratories in Santa Clara, Calif., to assess the mineral composition versus fruits such as blueberries, pomegranates, and cherries. The investigating lab recognized that because of its damaging effect on vital biological systems, oxidative stress has been implicated in more than 100 diseases (Ames et al., 1993). In the skin, they also recognized that oxidative stress is increased upon exposure to sunlight, pollutants and during inflammation, significantly contributing to dermal aging. Therefore, the concluded that compounds capable of shielding skin from the oxidative stress are extremely valuable for cosmetic and dermatological applications and the objective of the study was to measure the antioxidant potential of the mineral composition using oxygen radical absorbance capacity (ORAC) assay.

The test method for ORAC assay was performed according to the method described by Ou et al. (2001), with minor modifications. The ORAC assay measures the ability of antioxidant components to inhibit the decline in disodium fluorescein (FL) (Sigma-Aldrich, St Louis, Mo.) fluorescence that is induced by the peroxyl radical generator, 2′,2′-Azobis (2-amidinopropane)dihydrochloride (AAPH) (Wako Chemicals, Richmond, Va.). The reaction is conducted in the 96 well plate format. The reaction mixture contains test materials, FL (6.3×10⁻⁷M) and AAPH (1.28×10⁻¹M) in phosphate-buffered saline (PBS). The reaction is started by the addition of MPH. Fluorescence is measured at the emission length of 530 nm and excitation length of 485 nm using microplate fluorometer Cytofluor 2350 (Millipore). ORAC values are calculated from the quantification of the areas under the FL decay curve and are expressed as micromole (umol) Trolox equivalents (TE) per g.

[image]

The powdered mineral composition describe herein has been found to have good antioxidant activity amounting to 320 umole TE/g and compares favorably with fruits famous for their antioxidant activity, such as blueberries (Compound M is 15 times better g/g), pomegranates (10 times better) and sour cherry (20 times better, Blando et al., 2004).

References used in these test include Ames B N, Shigensaga M K, Hagen T M. Oxidants, antioxidants, and the degenerative diseases of aging. Proc Natl Acad Sci USA 1993, 90:7915-22. Blando F, Gerardi C, Nicoletti I. Sour Cherry (Prunus cerasus L) Anthocyanins as Ingredients for Functional Foods. Journal of Biomedicine and Biotechnology 2004, 5:253-258. Ou B, Hampsch-Woodill M, Prior R. L. Development and validation of an improved oxygen radical absorbance capacity assay using fluorescein as the fluorescent probe. J Agric Food Chem. 2001, 49:4619-4626.

The invention herein further describes methods for producing antioxidant skin care products that includes an unusually large number of minerals. Most particularly, the invention herein further describes methods of producing antioxidant skin cleansing compositions that include an unusually large number of minerals, described herein as mineral composition. Furthermore, the antioxidant properties displayed by the large number of minerals described herein is maximized when added into solution and immediately used on skin.

In addition, the antioxidant cleansing composition is devoid of any harsh sulfates or anionic surfactants such as sodium lauryl sulfate as these have been recognized as potential promoters of free radicals or free radical generators. Hence, the cleansing compositions described herein are sulfate-free.

The antioxidant skin cleaning composition can be applied topically to any part of the body, most particularly the area of the face. More importantly, the antioxidant cleansing compositions describe herein are designed to minimize the amount of oxidative stress that could be triggered by the action of cleansing.

The utilization of minerals as described herein and as utilized in a cleansing composition, is expected to synergistically or complimentarily have an antioxidant effect with other commonly used antioxidant ingredients to create a superior non-oxidizing antioxidant cleansing product.

The preferred embodiment of the inventions described herein describes an anhydrous, and most preferably a powdered antioxidant skin cleansing composition which preserves all antioxidant potential onto skin until the time of use.

The powdered antioxidant skin cleansing composition described herein can be applied directly to face first, and then spread with water to create the cleansing action before rinsing, or the product can be dispensed on the palm of the hand, mixed with water and then applied on the face before rinsing. Alternatively, either a dry or water pre-wetted brush can be used to apply the powdered skin cleansing composition. Hence, many packaging options can be used to house the cleansing composition, ranging from a sifter dispenser, to an open face jar with a spoon, to a jar with separate unit dose cup with a sponge or brush applicator.

Once the surface of the skin has been thoroughly cleansed with the powdered cleansing composition, rinsing with water to remove any loosened dirt, grime, and make-up is required before towel drying.

Furthermore, the invention described herein pertains to powdered skin cleansing composition that removes dirt, grime, make-up while conditioning in a skin compatible acidic pH that does not irritate dermal tissues when applied thereto.

Furthermore, the minerals described herein have been described as useful in providing exfoliation effects on skin, which compliments skin cleansing for renewing surface skin cells.

The powdered cleansing composition is preferably flow-able as a particulate ranging from a course semi-free flowing powder of 200-500 microns, to a fine free flowing powder of under 200 microns. The composition particle size could be physically modified to a desired micron size by physical means with the use of a grinder or pulverizer.

Furthermore, the powdered composition is substantially absent of water, containing less than 5% water, preferably less than 1%, most preferably less than 0.5%.

Use of antimicrobial agents or preservatives has been linked with potentially creating adverse skin reactions, such as irritation. Water is considered a medium for sustainable growth of microorganisms such as yeast, mold, and bacteria. Since the powdered cleansing composition described herein is substantially absent of water, there is a reduced need for antimicrobial agents or preservatives when compared to conventional water based cleansers. Therefore, the composition can be produced as a preservative-free or substantially free of preservatives. The presence of parabens in particular, as a class of effective preservatives widely used in personal care products mainly consisting of methylparaben, propylparaben, and butylparaben, are becoming ingredients of concern for safety. Since they are not required in the powdered cleansing composition, the powdered cleansing composition described here-in would be paraben-free.

The powdered cleansing composition delivers a pH to the skin of 4.0-6.2, preferable 5.0-6.0, most preferably 5.2-5.8 when combined with water during use which typically can be at a ratio of 20:1, preferably 10:1, most preferably 5:1 (water to cleanser ratio). A pH of 5.2-5.8 is an optimum pH for compatibility with the acid mantle of the skin.

The powdered cleansing composition is made up of a mineral based ingredient such as non-swelling and swelling clays. Kaolin, also known as aluminum silicates, is a non-swelling clay and is a known skin protectant in leave-on skin products. It is also recognized as an ideal absorbent of skin impurities. Use of kaolin in the powdered composition is preferably in a colloidal form and usage can range from levels of 1-99%, preferably 5-50%, most preferably 10-30%.

Other swelling clays such as Magnesium Aluminum Silicates can be used at concentrations of 1-30%, preferably 1-20%, most preferably 5-10%.

Plant-derived ingredients have been used in skin care applications since ancient times. Plant derived ingredient can provide a multitude of skin care benefits ranging from soothing, anti-inflammatory, antioxidant, astringent, antimicrobial, antiseptic, cleansing, conditioning, and many other beneficial skin effects. The powdered cleansing composition described herein utilizes plant derived ingredients for a broad range of benefits to enhance the performance of the overall powdered cleansing composition.

Among the plant derived ingredients utilized in the powdered cleansing composition is Rice Flour. Rice flour possesses gentle and natural exfoliant properties and its natural adhesive properties help absorb dirt and oils from pores as well as balance skin oils. Rice flour is gluten-free and non-allergic to skin, and can be utilized at concentrations of 1-99%, preferably 5-50%, most preferably 10-30%.

Alternatively, other starchy gluten-free flours can be utilized as well. For example, Tapioca flour has the ability to mitigate greasiness and absorb oil and can be used at concentrations of 1-99%, preferably 5-50%, most preferably 10-30%.

Other plant derived wheat and whey ingredients can be used at lower concentrations ranging from 0.1% to 10%. These could include Hydrolyzed Wheat Starch, Hydrolyzed Wheat Proteins, and Whey Mineral Proteins as examples. Colloidal Oatmeal can also be used for its oil absorbing properties, anti-irritant and anti-inflammatory properties. All of these ingredients contribute to the skin conditioning attributes of the powdered cleansing composition. Typically, a skin cleanser may contain a surfactant or detergent. For example, is it known typical facial cleansers can contain ingredients that reduce surface tension for facilitating the removal of Impurities present on skin. These surfactants can range in their chemical characteristics from non-ionic polysorbate 80, to strongly anionic sulfates such as Sodium Laureth Sulfate, to amphoteric surfactants such betaine derivatives. Many of these surfactants can be harsh on skin and irritating to eyes.

The powdered cleansing composition described herein is devoid of these harsh surfactants. Furthermore, the powdered cleansing composition described herein utilizes plant derived ingredients to reduce the surface tension of water to facilitate the removal of impurities present on the skin in a natural milder manner. Use of amino acid coconut derived surfactants are preferred in delivering mild surfactant qualities. Examples of these are glutamic acid coconut surfactants such as sodium cocoyl glutamate in powdered form. More specifically, plant derived ingredients containing saponins, which are natural detergents or surfactants found in plants, are preferred. The cleansing composition could contain plant-derived ingredients such as conditioners and as cleansing agents. It is known in the art that plant contain natural surfactants in plant cells that are capable of reducing surface tension, and even foaming. Plant saponins, for example, provide excellent surface activity and could be ideal for use in cleansers.

More specifically, preferred plant derived ingredients for the powdered cleansing composition should be among the richest in saponins to aid in reduction of surface tension. Examples of such plants are Yucca schidigera and Quillaja saponaria. Yucca schidigera which is found in the southwest United States and Mexico and Quillaja saponaria which comes from the Soap bark tree native to Chile, are preferred. Both plants stem from desert environments and quantitatively saponin rich.

Saponins contain a lipophilic nucleus which can be either a steroid or triterpinoid, with one or more water soluble carbohydrate side chain. In this regard, the surface activity reduction or surfactant properties of these plant components is attributed to both fat soluble and water soluble molecules all in each saponin molecule. Saponins are therefore a natural useful component in cleansing compositions to help remove impurities without the harshness of synthetic detergents. In addition, saponins, as a result of their surfactant attributes, have antimicrobial activity, and as part of their biological complexes may provide additional anti-inflammatory activity.

Since the powdered cleansing composition described herein is substantially free of water, a dry powdered form of the Yucca schidigera and the Quillaja saponaria is preferred. The preferred activity specifications for Yucca schidigera and Quillaja saponaria should range from 5-40% saponin content.

Yucca schidigera and Quillaja saponaria dried powder can be used in the powdered cleansing composition at concentrations of 0.1-25%, preferably 1-10%, and most preferably at concentrations of 2-5%. They are supplied by various vendors such as Garuda located in Lemon Cove, Calif., and Ultra Biologics located in Quebec, Canada.

The powdered cleansing composition described herein could also contain liquid plant derived ingredients which are either hydrophilic or lipophilic extracts or fractions as skin conditioning agents (i.e. emollients, conditioners, antioxidants, anti-inflammatory agents) or as aromatic essential oils. The plant derived ingredient could be derived from land or sea. Their usage can range from less than 20%, but preferably less than 10%, and most preferably less than 7.5%. Examples of these but not limited to are Coco nucifera (Coconut Oil), Aloe Barbadensis Leaf Extract, Soybean Extract, Camellia senensis, and Sage Essential Oil.

The powdered cleansing composition described herein could also contain other ingredients practiced in the art of skin care such as vitamins and antioxidants. Examples of these are Vitamin A, C, E, and Beta carotene.

### EXAMPLE 1

| | |
|---|---|
| Kaolin Colloidal, USP | 50.00 |
| Rice Flour | 20.00 |
| Dry Mineral Element Composition | 10.00 |
| Yucca schidigera | 10.00 |
| Quillaja saponaria | 10.00 |

### EXAMPLE 2

| | |
|---|---|
| Kaolin Colloidal, USP | 30.00 |
| Rice Flour | 27.00 |
| Tapioca Starch | 10.00 |
| Hydrolyzed Wheat Protein | 3.00 |
| Whey Mineral Protein | 4.00 |
| Dry Mineral Element Composition | 5.00 |
| Yucca schidigera | 15.00 |
| Quillaja saponaria | 6.00 |

### EXAMPLE 3

| | |
|---|---|
| Kaolin Colloidal, USP | 20.00 |
| Rice Flour | 20.00 |
| Tapioca Flour | 10.00 |
| Magnesium Aluminum Silicate | 8.00 |
| Hydroxylpropyl Wheat Starch | 4.00 |
| Hydrolyzed Wheat Protein | 3.00 |
| Whey Mineral Protein | 4.00 |
| Collodial Oatmeal | 2.00 |
| Dry Mineral Element Composition | 7.00 |
| Aloe Barbadenis | 3.00 |
| Yucca schidigera | 5.00 |
| Quillaja saponaria | 3.00 |
| Coconut Oil | 3.00 |
| Tocopherol Acetate | 0.50 |
| Beta Carotene | 0.50 |
| Camellia Senensis | 0.50 |
| Beta Glucam | 0.50 |
| Soybean Extract | 0.50 |
| Sodium Cocoyl Gluatmate | 5.00 |
| Sage Oil | 0.50 |

Having described the invention in such terms as to enable those skilled in the art to practice the invention, and having described the presently preferred embodiments thereof, we Claim:

1. A method of enhancing the ability of mammalian skin to combat free radicals, comprising the steps of applying to the skin a dry hygroscopic powder composition comprising a therapeutically effective amount of a mineral composition comprising
at least eight macro mineral elements;
at least sixty micro mineral elements; and,
at least ten rare earth elements.
